SEQUENCE LISTING (21 sequences)

SEQ ID NO: 1 - RNA, 22 nt, synthetic construct
  source 1..22: mol_type = other RNA
caaacaccattgtcacactcca

SEQ ID NO: 2 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 3 - RNA, 263 nt, synthetic construct
  source 1..263: mol_type = other RNA
gctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattg

SEQ ID NO: 4 - RNA, 153 nt, synthetic construct
  source 1..153: mol_type = other RNA
caaacaccattgtcacactccagctcgctttcttgctgtccaatttctattaaaggttcctttgttccctaagtccaactactaaactgggggatattatgaagggccttgagcatctggattctgcctaataaaaaacatttattttcattg

SEQ ID NO: 5 - RNA, 154 nt, synthetic construct
  source 1..154: mol_type = other RNA
gctcgctttcttgctgtccaatttctattaaaggttcctttgttccctaagtccaactactaaactgggggatattatgaagggccttgagcatctggattctgcctaataaaaaacatttattttcattgccaaacaccattgtcacactcca

SEQ ID NO: 6 - RNA, 285 nt, synthetic construct
  source 1..285: mol_type = other RNA
caaacaccattgtcacactccagctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattg

SEQ ID NO: 7 - RNA, 285 nt, synthetic construct
  source 1..285: mol_type = other RNA
gctcgctttcttgctgtccaatttctattaaaggttcctttgttccctaagtccaactactaaactgggggatattatgaagggccttgagcatctggattctgcctaataaaaaacatttattttcattgccaaacaccattgtcacactccagctcgctttcttgctgtccaatttctattaaaggttcctttgttccctaagtccaactactaaactgggggatattatgaagggccttgagcatctggattctgcctaataaaaaacatttattttcattg

SEQ ID NO: 8 - RNA, 285 nt, synthetic construct
  source 1..285: mol_type = other RNA
gctcgctttcttgctgtccaatttctattaaaggttcctttgttccctaagtccaactactaaactgggggatattatgaagggccttgagcatctggattctgcctaataaaaaacatttattttcattgcgctcgctttcttgctgtccaatttctattaaaggttcctttgttccctaagtccaactactaaactgggggatattatgaagggccttgagcatctggattctgcctaataaaaaacatttattttcattgcaaacaccattgtcacactcca

SEQ ID NO: 9 - RNA, 136 nt, synthetic construct
  source 1..136: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 10 - RNA, 272 nt, synthetic construct
  source 1..272: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctccctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 11 - RNA, 294 nt, synthetic construct
  source 1..294: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaaacaccattgtcacactccactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 12 - RNA, 294 nt, synthetic construct
  source 1..294: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctccctggtactgcatgcacgcaatgctagctgcccctttcccgtcctgggtaccccgagtctcccccgacctcgggtcccaggtatgctcccacctccacctgccccactcaccacctctgctagttccagacacctcccaaacaccattgtcacactcca

SEQ ID NO: 13 - RNA, 142 nt, synthetic construct
  source 1..142: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 14 - RNA, 284 nt, synthetic construct
  source 1..284: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 15 - RNA, 306 nt, synthetic construct
  source 1..306: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacacccaaacaccattgtcacactccacaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 16 - RNA, 306 nt, synthetic construct
  source 1..306: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacacccaagcacgcagcaatgcagctcaaaacgcttagcctagccacacccccacgggaaacagcagtgattaacctttagcaataaacgaaagtttaactaagctatactaaccccagggttggtcaatttcgtgccagccacacccaaacaccattgtcacactcca

SEQ ID NO: 17 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 18 - RNA, 300 nt, synthetic construct
  source 1..300: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaaacaccattgtcacactccacaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 19 - RNA, 300 nt, synthetic construct
  source 1..300: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacccaaacaccattgtcacactcca

SEQ ID NO: 20 - RNA, 1653 nt, synthetic construct
  source 1..1653: mol_type = other RNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaaaagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 21 - RNA, 85 nt, synthetic construct
  source 1..85: mol_type = other RNA
ccttagcagagctgtggagtgtgacaatggtgtttgtgtctaaactatcaaacgccattatcacactaaatagctactgctaggc

(19) United States
(12) Patent Application Publication
(10) Pub. No.: US 2025/0276096 A1
(43) Pub. Date: Sep. 4, 2025

(54) mRNA DRUG THAT IS LESS EXPRESSED IN THE LIVER AFTER IN VIVO DELIVERY AND PREPARATION METHOD THEREOF

(71) Applicants: Beijing Neocurna Biotechnology Corporation, Beijing (CN); Neocura Bio-Medical Technology Co., Ltd, Guangdong (CN); Shenzhen Neocurna Biotechnology Corporation, Guangdong (CN)

(72) Inventors: Youdong PAN, Beijing (CN); Lu Qian, Beijing (CN); Yijue Xue, Beijing (CN); Ren Zhu, Beijing (CN); Ji Wan, Beijing (CN); Zhao Zhao, Beijing (CN); Yi Wang, Beijing (CN)

(73) Assignees: Beijing Neocurna Biotechnology Corporation, Beijing (CN); Neocura Bio-Medical Technology Co., Ltd, Guangdong (CN); Shenzhen Neocurna Biotechnology Corporation, Guangdong (CN)

(21) Appl. No.: 19/183,930

(22) Filed: Apr. 21, 2025

Related U.S. Application Data
(63) Continuation of application No. PCT/CN2023/077347, filed on Feb. 21, 2023.

(30) Foreign Application Priority Data
Oct. 27, 2022 (CN) 202211329780.5

(51) Int. Cl. A61K 48/00

(52) U.S. Cl. CPC A61K 48/0058

(57) ABSTRACT

An mRNA drug, which reduces expression in the liver after in vivo delivery, and a preparation method thereof. The preparation method comprises: establishing double 3′ UTR in a target mRNA, and introducing a miR-122 binding site between the double 3′ UTR, so as to effectively reduce the expression of the mRNA drug, which is delivered by means of LNP, in the liver, thereby realizing a specific inhibitory effect of miR-122 while retaining an enhancement effect of miR-122 on mRNA stability and translation efficiency. The mRNA drug can effectively reduce the expression of mRNA in the liver, realizing the efficient expression of the mRNA drug in non-hepatocellular targeted cells or tissues and increasing the effective non-hepatocyte tropism of the mRNA drug.

### CROSS-REFERENCE TO RELATED APPLICATION

This application is a continuation of international application of PCT application serial no. PCT/CN2023/077347, filed on Feb. 21, 2023, which claims the priority benefit of China application no. 202211329780.5, filed on Oct. 27, 2022. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Apr. 11, 2025, is named 154867US-sequencing_listing and is 25,608 bytes in size.

### BACKGROUND

### Technical Field

The present disclosure relates to the field of biomedical technology, particularly to a method for preparing mRNA drug that is less expressed in the liver after in vivo delivery and preparation method thereof.

### Description of Related Art

Due to the influence of COVID-19, mRNA has entered people's vision as a new therapeutic agent for preventing and treating various diseases. Messenger RNA (mRNA) is a template for guiding protein synthesis and serves as a messenger that transmits genetic information from DNA to proteins. The main sequence of mature mRNA is the coding region with non-coding regions at its both upstream 5′end and downstream 3′ end. There are 5′ caps and 3′ tails at both ends of eukaryotic mRNA molecules. The 5′cap structure plays an important role in the stability and translation of mRNA, as it blocks the 5′ end and prevents it from being hydrolyzed by exonucleases; as a recognition signal of the protein synthesis system, it is recognized and bound by the cap binding protein (eIF-4E), which promotes the binding of mRNA to ribosomal subunits and thereby initiates the translation process. The 5′untranslated region (5′ UTR) is a shorter sequence between the cap and the start codon of the encoding region, which includes the sequence that marks the start of translation. The coding region, also known as the Open Reading Frame (ORF), starts from the start codon (AUG) and ends at the stop codon (UAG, UGA, UAA), encoding the primary structure of a protein. The secondary structure and codon selection in the encoding area may both affect translation efficiency. Excessive secondary structures and rare codons can slow down translation speed, therefore, when genetic engineering is used to express proteins, it will be optimized based on the host's codon preferences. The 3′untranslated region (3′ UTR) is the transcription sequence after codon termination and also participates in translation regulation. For example, many microRNAs can bind to the 3′ untranslated region of the target gene mRNA, downregulating the expression of the target gene by degrading or inhibiting the translation process. Mature mRNA typically has a poly A tail with a length of 20-200 bases at the 3′end, which can prevent exonuclease degradation. The tail structure is also related to the translation process and its regulation. For example, polyadenylation binding protein (PABP) can bind to the tail and further interact with various proteins such as eIF4G, eIF4B, Paip-1, forming circular complexes that participate in translation initiation process and mRNA stability regulation process.

mRNA molecules are large and negatively charged, making it difficult to passively cross negatively charged cell membranes. In addition, RNA enzymes in blood and tissues can rapidly degrade mRNA, and induce innate immune responses. In order to achieve therapeutic effects, mRNA requires a safe, effective, and stable delivery system to protect nucleic acids from degradation in the body, while delivering mRNA to specific target cells and producing sufficient protein. An mRNA drug delivery system based on lipids, polymers, dendritic molecules, and natural membranes has been successfully developed to deliver mRNA to target cells. The most commonly used mRNA vaccine delivery technologies currently include lipid nanoparticles (LNP), cationic lipid complexes (lipoplex, LPX), lipid polyplexes (LPP), polymer nanoparticles (PNP), inorganic nanoparticles (INP), and cationic nanoemulsions (CNE), etc. With the approval of two types of mRNA vaccines for vaccination against COVID-19, LNP has become the most popular delivery technology at present. The application of mRNA induced transient protein expression is not only applicable in the field of vaccines for infectious diseases, but also provides new avenues for cancer vaccines, protein replacement therapies, and gene editing for rare genetic diseases.

Currently, most mRNA/LNP delivery methods exhibit high liver expression during systemic administration, but more application scenarios of mRNA drugs require non-liver-targeted mRNA delivery, especially for drugs with hepatotoxicity or systemic toxicity. However, suitable methods need to be found to eliminate mRNA delivery to the liver or block mRNA expression in the liver when the drugs achieve local or target tissue organ delivery expression.

microRNA (miRNA) is a group of non-coding RNAs with a length of approximately 20 to 23 nucleotides encoded by the genome. It mainly acts on the mRNA 3′UTR region of target gene by guiding the silencing complex (RISC) through base pairing to degrade mRNA or hinder its translation. miRNA is highly conserved in species evolution, and its expression exhibits tissue specificity and temporal specificity. Due to the high specific expression of miR-122 in hepatocytes, miR-122 can be used in the optimization design of mRNA 3′UTR. The miR-122 binding site was introduced into the preferred 3′UTR sequence, resulting in mRNA that is more easily degraded in hepatocytes than in other types of cells, thereby reducing its expression in normal hepatocytes and enhancing its effective non-liver cell tendency.

At present, there is no in-depth research on how to use microRNA-122 to reduce the expression of mRNA drugs in the liver after delivery into the body.

### SUMMARY

Based on this, the purpose of the present disclosure is to provide an mRNA drug that is less expressed in the liver after in vivo delivery and a preparation method thereof.

The present disclosure includes the following technical solutions.

The first aspect of the present disclosure is to provide an mRNA drug that is less expressed in the liver after delivery into the body.

An mRNA drug, which is less expressed in the liver after in vivo delivery, comprising mRNA and a drug carrier, wherein a 3′UTR component of the mRNA comprises two identical or different 3′ UTR sequences, and a miR-122 binding site is inserted between the two 3′UTR sequences.

The second aspect of the present disclosure is to provide a method for preparing the mRNA drugs.

A method for preparing an mRNA drug, comprising including two identical or different 3′ UTR sequences in the 3′ UTR component of mRNA, inserting a miR-122 binding site between the connections of the two 3′ UTR sequences, and obtaining mRNA for preparing an mRNA drug.

The third aspect of the disclosure is to provide a method for reducing mRNA drug expression in the liver after delivery into the body.

A method for reducing the expression of an mRNA drug in the liver after delivery into the body, wherein the 3′UTR component of mRNA in the mRNA drug comprises two identical or different 3′ UTR sequences, and a miR-122 binding site is inserted between the two 3′UTR sequences.

The present disclosure mainly aims to use liver-specifically expressed miR-122 and its binding site sequence to screen for the optimal miR-122 binding site insertion method in mRNA 3′ UTR, that is, to select a 3′ UTR sequence and insert a miR-122 binding site between the double copy UTR to achieve the specific inhibitory effect of miR-122 while retaining its enhancing effect on mRNA stability and translation efficiency. The technology described in the present disclosure can effectively reduce the expression of mRNA drugs in the liver, and achieve efficient expression of mRNA drugs in other cells or tissues that are not targeted by hepatocytes. Based on the above, the present disclosure provides an mRNA drug that reduces expression in the liver after delivery to the body, including the insertion of a miR-122 binding site between the target mRNA double copy 3′ UTR, which can effectively reduce the expression of the delivered mRNA drug in the liver. The obtained mRNA is more easily degraded in hepatocytes than in other cell types, thereby increasing its effective non hepatic properties.

### BRIEF DESCRIPTION OF THE DRAWINGS

FIG. 1. Schematic diagram of different UTR₁₂₂ sequence designs based on HBB 3′UTR.

FIG. 2. Expressions of miR-122 in several kinds of liver cancer cell lines.

FIG. 3. Response inhibition of miR-122 mimics by different HBB UTR₁₂₂.

FIG. 4. Schematic diagram of UTR₁₂₂ sequence design based on AES and mtRNR1 3′UTR.

FIG. 5. Response inhibition of miR-122 mimics by UTR₁₂₂ based on AES and mtRNR1 3′UTR.

FIG. 6. Inhibition of miR-122 mimics on Fluc mRNA in HepG2 and Huh7 cells carrying different UTR₁₂₂.

FIG. 7. In vivo imaging results of Fluc-2XHBB/LNP and Fluc-2XHBB₁₂₂ intravenous injection in mice.

FIG. 8. Fluorescence intensity statistics of in vivo imaging of Fluc-2XHBB/LNP and Fluc-2XHBB₁₂₂ intravenous injection in mice.

FIG. 9. In vivo imaging results of Fluc-2XHBB₁₂₂/LNP intravenous injection and subcutaneous injection in mice.

FIG. 10. Fluorescence intensity of bioluminescence in vivo imaging of mice injected intravenously and subcutaneously with Fluc-2XHBB₁₂₂/LNP.

FIG. 11. Imaging results of intratumoral injection in mice.

FIG. 12. Statistical chart of bioluminescence intensity in vivo imaging of intratumoral injection in mice.

FIG. 13. Schematic diagram of GFP-2XHBB and GFP-2XHBB₁₂₂ sequence design.

FIG. 14. Fluorescence microscopy results of transfected 293T cells.

FIG. 15. Statistical results of 293T cells through flow cytometry.

FIG. 16. Fluorescence microscopy results of transfected CHO cells.

FIG. 17. Statistical results of transfected CHO cells through flow cytometry.

### DESCRIPTION OF THE EMBODIMENTS

In order to facilitate the understanding of the present disclosure, a more comprehensive description about the present disclosure is given below. The present disclosure can be implemented in many different forms, and is not limited to the embodiments described herein. On the contrary, these embodiments are provided to make the understanding of the disclosure more thorough and comprehensive.

The experimental methods without specifying specific conditions in the following embodiments generally follow conventional conditions, such as those described in Sambrook et al., Molecular Cloning: A Laboratory Manual, 2013 (New York: Cold Spring Harbor Laboratory Press, 1989), or the conditions recommended by the manufacturers. The various commonly used chemical reagents used in the embodiments are all commercially available products.

Unless otherwise defined, all technical and scientific terms used herein have the same meaning as commonly understood by the skilled in the art of the present disclosure. The terms used in the description of the present disclosure are for describing specific embodiments only and are not intended to limit the present disclosure. The term “and/or” used in the present disclosure includes any and all combinations of one or more associated listed items.

The 3′UTR region in eukaryotes can affect mRNA stability, microRNA-mediated degradation, and protein translation efficiency. There is an optimal length requirement for 3′UTR, because mRNA with longer 3′UTR has a shorter half-life, while mRNA with shorter 3′UTR has lower translation efficiency. The 3′UTR commonly used in mRNA therapy is derived from human α- and β-globins (HBA and HBB), which has been widely used to deliver mRNA to various cell types. BioNTech SE screened naturally occurring 3′UTRs using SELEX technology and functionally determined the optimal combination of double-copy UTR elements (dUTR) AES-mtRNR1 and mtRNR1-AES [mtRNR1 (Mitochondrially Encoded 12S rRNA, non-coding 12S rRNA encoded by mitochondria); AES (Amino terminal enhancer of split, a member of transcription factor Groucho/TLE family]. However, they found that, in fact, since the factors regulating mRNA stability are cell type specific, there may be elements based on AES-mtRNR1 that are not superior to the conventionally used 2HBB 3′UTR.

The location and number of miRNA binding sites on mRNA 3′UTR may affect mRNA transcriptional stability and gene delivery in specific cells. The inventor found that inserting the miR-122 binding site sequence into the appropriate position of the double copy 3′UTR (between the two 3′UTRs) can achieve efficient non-hepatocyte expression without weakening the effect of the 3′UTR itself on mRNA transcriptional stability, but also effectively reduce the expression of the delivered mRNA drug in the normal liver.

In some embodiments of the present disclosure, it is related to an mRNA drug, which is less expressed in the liver after in vivo delivery, comprising mRNA and a drug carrier, wherein a 3′UTR component of the mRNA comprises two identical or different 3′ UTR sequences, and a miR-122 binding site is inserted between the two 3′UTR sequences.

In some embodiments, the sequence of the miR-122 binding site as set forth in SEQ ID NO: 1.

In some embodiments, the 3′UTR sequence is at least one of the 3′ UTR sequence of human hemoglobin βsubunit, an AES element, and a mtRNR1 element.

In some embodiments, the 3′ UTR sequence of the human hemoglobin β subunit as set forth in SEQ ID NO:2.

In some embodiments, the AES element as set forth in SEQ ID NO:9.

In some embodiments, the mtRNR1 element as set forth in SEQ ID NO:13.

In some embodiments, the 3′ UTR component is composed of the AES element, the miR-122 binding site, and the AES element.

In some embodiments, the 3′ UTR component is composed of the AES element, the miR-122 binding site, and the mtRNR1 element.

In some embodiments, the 3′ UTR component is composed of the mtRNR1 element, the miR-122 binding site, and the mtRNR1 element.

In some embodiments, the 3′ UTR component is composed of the 3′ UTR sequence of the human hemoglobin β subunit, the miR-122 binding site, and the 3′ UTR sequence of the human hemoglobin β subunit.

In some embodiments, the drug carrier can be various carriers of known mRNA drugs, such as a lipid nanoparticle, a complex and a polymer nanoparticle, an exosome, or a biological microvesicle, etc.

In some embodiments, the mRNA drug is an mRNA vaccine.

In some of these embodiments, the present disclosure found through experimental data that: according to the small animal in vivo imaging system, it can be observed and analyzed the location and intensity of different luminescent signals at different times and different imaging positions in mice under different intravenous administration methods. After intravenous administration, the bioluminescence signals of mice in each group were mainly distributed in the liver area, and the luminescence signals gradually weakened with time. In the Fluc/LNP group, the drug was basically metabolized and cleared at 48 hours imaging, while in the Fluc-HBB 122/LNP group, the drug was basically metabolized and cleared at 24 hours imaging.

The imaging intensity of animals at different times and different positions was compared and analyzed based on the analysis data of luminescence signal intensity through small animal in vivo imaging system. The bioluminescence signal values of the Fluc/LNP group were significantly higher than those of the Fluc-HBB122/LNP group at each time point (p<0.05).

According to the small animal in vivo imaging system, it can be observed and analyzed the intensity of different luminescent signals in mice at different times under intravenous and subcutaneous injection administration methods. The bioluminescence signals of mice in each group were mainly distributed in the liver area, and tended to weaken gradually over time. The abdominal imaging fluorescence signals of the intravenous injection group were close to the lateral imaging fluorescence signals, while the abdominal imaging fluorescence signals of the subcutaneous injection group were significantly lower than the lateral imaging fluorescence signals.

The imaging intensity of animals at different times and different body positions were compared and analyzed based on the analysis data of luminescence signal intensity through a small animal in vivo imaging system. The bioluminescence signals of mice in each group gradually weakened over time. The abdominal imaging fluorescence signals of the intravenous injection group were similar to those of the lateral imaging fluorescence signal, while the abdominal imaging fluorescence signals of the subcutaneous injection group were significantly lower than those of the lateral imaging fluorescence signal.

According to the small animal in vivo imaging system, it can be observed and analyzed the position and intensity of different luminescent signals in mice 6 hours after intratumoral injection. The bioluminescence signals of mice in each group were mainly distributed in tumor and liver regions. The fluorescence signals of abdominal and lateral imaging in the Fluc-2X HBB122/LNP group were lower than those in the Fluc-2XHBB/LNP group. The Fluc-2XHBB₁₂₂/LNP group had stronger luminescence signals at the tumor site and weaker luminescence signals at the liver site. The Fluc-2XHBB/LNP group showed strong signals in both tumor and liver regions, and with the same luminescent signals intensity at tumor sites in both groups.

In some embodiments, the experimental data of the present disclosure also found that, based on the analysis data of the luminescence signal intensity by the small animal in vivo imaging instrument, the position and intensity of different luminescence signals after 6 hours of intratumoral administration in animals were compared and analyzed. The fluorescence signals of the abdominal and lateral imaging in the Fluc-2XHBB122/LNP group were lower than those in the Fluc-2XHBB/LNP group, and the luminescence signal in the tumor site was stronger and the luminescence signal in the liver site was weaker in the Fluc-2XHBB122/LNP group; the Fluc-2XHBB/LNP group showed strong signals in both tumor and liver regions, with the same intensity of luminescent signals in both tumor sites. The luminescence signal of the liver in the Fluc-2XHBB/LNP group was 13 times of that in the Fluc-2XHBB122/LNP group.

Observation under a fluorescence microscope showed that as the miR-122 gene content increased, there was no change in the expression level of GFP in 293T cells transfected with GFP-2XHBB sequence, while the expression level of GFP in 293T cells transfected with GFP-2XHBB122 sequence gradually decreased.

The GFP expression level in 293T cells was detected by flow cytometry when 25 nM miR-122 gene was added. The GFP signal of 293T cells transfected with GFP-2XHBB sequence was significantly stronger than that of 293T cells transfected with GFP-2XHBB122 sequence.

Observation under a fluorescence microscopy showed, with the increase of miR-122 gene content, the expression level of GFP in CHO-K1 cells transfected with GFP-2XHBB sequence remained unchanged, while the expression level of GFP gradually decreased in CHO-K1 cells transfected with GFP-2XHBB122 sequence.

The GFP expression level of CHO-K1 cells with different levels of miR-122 gene were detected by flow cytometry. The GFP signal of CHO-K1 cells transfected with GFP-2XHBB sequence showed no significant change, while the GFP signal of CHO-K1 cells transfected with GFP-2XHBB122 sequence gradually weakened with the increase of miR-122 gene content.

Further detailed explanation of the present disclosure is provided below with reference to specific embodiments.

### Embodiment 1

The mRNA in the present disclosure was synthesized through in vitro transcription using a reagent kit. The sequence encoding Fluc was a publicly available sequence. mRNA encoding Fluc was used for in vivo evaluation in mice. In the mRNA synthesis, in addition to introducing miR-122 binding site into the 3′UTR, different sites of the mRNA were modified, including capping modification at the 5′end and adding more than 100 of poly-A molecules at the 3′ end, to enhance the stability of mRNA transcribed in vitro. The designed coding region sequence can be replaced with epitopes of different target genes as needed, making it suitable for different mRNA drug designs. In addition, studies have shown that modified nucleotide, such as using pseudo-UTP to replace conventional nucleotide in mRNA. It can enhance the stability of mRNA while reducing the stress response in vivo.

TABLE 1

| SEQ | | Sequence ( 5′-3′); The underline |
|---|---|---|
| ID | | represents the binding site of |
| NO: | Name | miR-122 |
| 1 | binding | CAAACACCAUUGUCACACUCCA |
| | site | |
| | of miR- | |
| | 122 | |
| 2 | HBB | GCUCGCUUUCUUGCUGUCCAAUUUCUAUUAAAGGUUC |
| | 3′UTR | CUUUGUUCCCUAAGUCCAACUACUAAACUGGGGGAUA |
| | | UUAUGAAGGGCCUUGAGCAUCUGGAUUCUGCCUAAUA |
| | | AAAAACAUUUAUUUUCAUUGC |
| 3 | HBB-HBB | GCUCGCUUUCUUGCUGUCCAAUUUCUAUUAAAGGUUC |
| | 3′UTR | CUUUGUUCCCUAAGUCCAACUACUAAACUGGGGGAUA |
| | | UUAUGAAGGGCCUUGAGCAUCUGGAUUCUGCCUAAUA |
| | | AAAAACAUUUAUUUUCAUUGCGCUCGCUUUCUUGCUG |
| | | UCCAAUUUCUAUUAAAGGUUCCUUUGUUCCCUAAGUC |
| | | CAACUACUAAACUGGGGGAUAUUAUGAAGGGCCUUGA |
| | | GCAUCUGGAUUCUGCCUAAUAAAAAACAUUUAUUUUC |
| | | AUUG |
| 4 | 122-HBB | CAAACACCAUUGUCACACUCCAGCUCGCUUUCUUGCUG |
| | 3′UTR | UCCAAUUUCUAUUAAAGGUUCCUUUGUUCCCUAAGUC |
| | | CAACUACUAAACUGGGGGAUAUUAUGAAGGGCCUUGA |
| | | GCAUCUGGAUUCUGCCUAAUAAAAAACAUUUAUUUUC |
| | | AUUG |
| 5 | HBB-122 | GCUCGCUUUCUUGCUGUCCAAUUUCUAUUAAAGGUUC |
| | 3′UTR | CUUUGUUCCCUAAGUCCAACUACUAAACUGGGGGAUA |
| | | UUAUGAAGGGCCUUGAGCAUCUGGAUUCUGCCUAAUA |
| | | AAAAACAUUUAUUUUCAUUGCCAAACACCAUUGUCAC |
| | | ACUCCA |
| 6 | 122- | CAAACACCAUUGUCACACUCCAGCUCGCUUUCUUGCUG |
| | HBB- | UCCAAUUUCUAUUAAAGGUUCCUUUGUUCCCUAAGUC |
| | HBB | CAACUACUAAACUGGGGGAUAUUAUGAAGGGCCUUGA |
| | 3′UTR | GCAUCUGGAUUCUGCCUAAUAAAAAACAUUUAUUUUC |
| | | AUUGCGCUCGCUUUCUUGCUGUCCAAUUUCUAUUAAA |
| | | GGUUCCUUUGUUCCCUAAGUCCAACUACUAAACUGGG |
| | | GGAUAUUAUGAAGGGCCUUGAGCAUCUGGAUUCUGCC |
| | | UAAUAAAAAACAUUUAUUUUCAUUG |
| 7 | HBB- | GCUCGCUUUCUUGCUGUCCAAUUUCUAUUAAAGGUUC |
| | 122- | CUUUGUUCCCUAAGUCCAACUACUAAACUGGGGGAUA |
| | HBB | UUAUGAAGGGCCUUGAGCAUCUGGAUUCUGCCUAAUA |
| | 3′UTR | AAAAACAUUUAUUUUCAUUGCCAAACACCAUUGUCAC |
| | | ACUCCAGCUCGCUUUCUUGCUGUCCAAUUUCUAUUAAA |
| | | GGUUCCUUUGUUCCCUAAGUCCAACUACUAAACUGGG |
| | | GGAUAUUAUGAAGGGCCUUGAGCAUCUGGAUUCUGCC |
| | | UAAUAAAAAACAUUUAUUUUCAUUG |
| 8 | HBB- | GCUCGCUUUCUUGCUGUCCAAUUUCUAUUAAAGGUUC |
| | HBB- | CUUUGUUCCCUAAGUCCAACUACUAAACUGGGGGAUA |
| | 122 | UUAUGAAGGGCCUUGAGCAUCUGGAUUCUGCCUAAUA |
| | 3′UTR | AAAAACAUUUAUUUUCAUUGCGCUCGCUUUCUUGCUG |
| | | UCCAAUUUCUAUUAAAGGUUCCUUUGUUCCCUAAGUC |
| | | CAACUACUAAACUGGGGGAUAUUAUGAAGGGCCUUGA |
| | | GCAUCUGGAUUCUGCCUAAUAAAAAACAUUUAUUUUC |
| | | AUUGCAAACACCAUUGUCACACUCCA |
| 9 | AES | CUGGUACUGCAUGCACGCAAUGCUAGCUGCCCCUUUCC |
| | 3′UTR | CGUCCUGGGUACCCCGAGUCUCCCCCGACCUCGGGUCC |
| | | CAGGUAUGCUCCCACCUCCACCUGCCCCACUCACCACC |
| | | UCUGCUAGUUCCAGACACCUCC |
| 10 | AES-AES | CUGGUACUGCAUGCACGCAAUGCUAGCUGCCCCUUUCC |
| | 3′UTR | CGUCCUGGGUACCCCGAGUCUCCCCCGACCUCGGGUCC |
| | | CAGGUAUGCUCCCACCUCCACCUGCCCCACUCACCACC |
| | | UCUGCUAGUUCCAGACACCUCCCUGGUACUGCAUGCAC |
| | | GCAAUGCUAGCUGCCCCUUUCCCGUCCUGGGUACCCCG |
| | | AGUCUCCCCCGACCUCGGGUCCCAGGUAUGCUCCCACC |
| | | UCCACCUGCCCCACUCACCACCUCUGCUAGUUCCAGAC |
| | | ACCUCC |
| 11 | AES- | CUGGUACUGCAUGCACGCAAUGCUAGCUGCCCCUUUCC |
| | 122- | CGUCCUGGGUACCCCGAGUCUCCCCCGACCUCGGGUCC |
| | AES | CAGGUAUGCUCCCACCUCCACCUGCCCCACUCACCACC |
| | 3′UTR | UCUGCUAGUUCCAGACACCUCCCAAACACCAUUGUCAC |
| | | ACUCCACUGGUACUGCAUGCACGCAAUGCUAGCUGCCC |
| | | CUUUCCCGUCCUGGGUACCCCGAGUCUCCCCCGACCUC |
| | | GGGUCCCAGGUAUGCUCCCACCUCCACCUGCCCCACUC |
| | | ACCACCUCUGCUAGUUCCAGACACCUCC |
| 12 | AES- | CUGGUACUGCAUGCACGCAAUGCUAGCUGCCCCUUUCC |
| | AES- | CGUCCUGGGUACCCCGAGUCUCCCCCGACCUCGGGUCC |
| | 122 | CAGGUAUGCUCCCACCUCCACCUGCCCCACUCACCACC |
| | 3′UTR | UCUGCUAGUUCCAGACACCUCCCUGGUACUGCAUGCAC |
| | | GCAAUGCUAGCUGCCCCUUUCCCGUCCUGGGUACCCCG |
| | | AGUCUCCCCCGACCUCGGGUCCCAGGUAUGCUCCCACC |
| | | UCCACCUGCCCCACUCACCACCUCUGCUAGUUCCAGAC |
| | | ACCUCCCAAACACCAUUGUCACACUCCA |
| 13 | mtRNR1 | CAAGCACGCAGCAAUGCAGCUCAAAACGCUUAGCCUAG |
| | 3′UTR | CCACACCCCCACGGGAAACAGCAGUGAUUAACCUUUAG |
| | | CAAUAAACGAAAGUUUAACUAAGCUAUACUAACCCCA |
| | | GGGUUGGUCAAUUUCGUGCCAGCCACACC |
| 14 | mtRNR1- | CAAGCACGCAGCAAUGCAGCUCAAAACGCUUAGCCUAG |
| | mtRNR1 | CCACACCCCCACGGGAAACAGCAGUGAUUAACCUUUAG |
| | 3′UTR | CAAUAAACGAAAGUUUAACUAAGCUAUACUAACCCCA |
| | | GGGUUGGUCAAUUUCGUGCCAGCCACACCCAAGCACGC |
| | | AGCAAUGCAGCUCAAAACGCUUAGCCUAGCCACACCCC |
| | | CACGGGAAACAGCAGUGAUUAACCUUUAGCAAUAAAC |
| | | GAAAGUUUAACUAAGCUAUACUAACCCCAGGGUUGGU |
| | | CAAUUUCGUGCCAGCCACACC |
| 15 | mtRNR1- | CAAGCACGCAGCAAUGCAGCUCAAAACGCUUAGCCUAG |
| | 122- | CCACACCCCCACGGGAAACAGCAGUGAUUAACCUUUAG |
| | mtRNR1 | CAAUAAACGAAAGUUUAACUAAGCUAUACUAACCCCA |
| | 3′UTR | GGGUUGGUCAAUUUCGUGCCAGCCACACCCAAACACCA |
| | | UUGUCACACUCCACAAGCACGCAGCAAUGCAGCUCAAA |
| | | ACGCUUAGCCUAGCCACACCCCCACGGGAAACAGCAGU |
| | | GAUUAACCUUUAGCAAUAAACGAAAGUUUAACUAAGC |
| | | UAUACUAACCCCAGGGUUGGUCAAUUUCGUGCCAGCCA |
| | | CACC |
| 16 | mtRNR1- | CAAGCACGCAGCAAUGCAGCUCAAAACGCUUAGCCUAG |
| | mtRNR1- | CCACACCCCCACGGGAAACAGCAGUGAUUAACCUUUAG |
| | 122 | CAAUAAACGAAAGUUUAACUAAGCUAUACUAACCCCA |
| | 3′UTR | GGGUUGGUCAAUUUCGUGCCAGCCACACCCAAGCACGC |
| | | AGCAAUGCAGCUCAAAACGCUUAGCCUAGCCACACCCC |
| | | CACGGGAAACAGCAGUGAUUAACCUUUAGCAAUAAAC |
| | | GAAAGUUUAACUAAGCUAUACUAACCCCAGGGUUGGU |
| | | CAAUUUCGUGCCAGCCACACCCAAACACCAUUGUCACA |
| | | CUCCA |
| 17 | AES- | CUGGUACUGCAUGCACGCAAUGCUAGCUGCCCCUUUCC |
| | mtRNR1 | CGUCCUGGGUACCCCGAGUCUCCCCCGACCUCGGGUCC |
| | 3′UTR | CAGGUAUGCUCCCACCUCCACCUGCCCCACUCACCACC |
| | | UCUGCUAGUUCCAGACACCUCCCAAGCACGCAGCAAUG |
| | | CAGCUCAAAACGCUUAGCCUAGCCACACCCCCACGGGA |
| | | AACAGCAGUGAUUAACCUUUAGCAAUAAACGAAAGUU |
| | | UAACUAAGCUAUACUAACCCCAGGGUUGGUCAAUUUC |
| | | GUGCCAGCCACACC |
| 18 | AES- | CUGGUACUGCAUGCACGCAAUGCUAGCUGCCCCUUUCC |
| | 122- | CGUCCUGGGUACCCCGAGUCUCCCCCGACCUCGGGUCC |
| | mtRNR1 | CAGGUAUGCUCCCACCUCCACCUGCCCCACUCACCACC |
| | 3′UTR | UCUGCUAGUUCCAGACACCUCCCAAACACCAUUGUCAC |
| | | ACUCCACAAGCACGCAGCAAUGCAGCUCAAAACGCUUA |
| | | GCCUAGCCACACCCCCACGGGAAACAGCAGUGAUUAAC |
| | | CUUUAGCAAUAAACGAAAGUUUAACUAAGCUAUACUA |
| | | ACCCCAGGGUUGGUCAAUUUCGUGCCAGCCACACC |
| 19 | AES- | CUGGUACUGCAUGCACGCAAUGCUAGCUGCCCCUUUCC |
| | mtRNR1- | CGUCCUGGGUACCCCGAGUCUCCCCCGACCUCGGGUCC |
| | 122 | CAGGUAUGCUCCCACCUCCACCUGCCCCACUCACCACC |
| | 3′UTR | UCUGCUAGUUCCAGACACCUCCCAAGCACGCAGCAAUG |
| | | CAGCUCAAAACGCUUAGCCUAGCCACACCCCCACGGGA |
| | | AACAGCAGUGAUUAACCUUUAGCAAUAAACGAAAGUU |
| | | UAACUAAGCUAUACUAACCCCAGGGUUGGUCAAUUUC |
| | | GUGCCAGCCACACCCAAACACCAUUGUCACACUCCA |
| 20 | Fluc | ATGGAAGATGCCAAAAACATTAAGAAGGGCCCAGCGCC |
| | CDS | ATTCTACCCACTCGAAGACGGGACCGCCGGCGAGCAGCT |
| | | GCACAAAGCCATGAAGCGCTACGCCCTGGTGCCCGGCA |
| | | CCATCGCCTTTACCGACGCACATATCGAGGTGGACATTA |
| | | CCTACGCCGAGTACTTCGAGATGAGCGTTCGGCTGGCAG |
| | | AAGCTATGAAGCGCTATGGGCTGAATACAAACCATCGG |
| | | ATCGTGGTGTGCAGCGAGAATAGCTTGCAGTTCTTCATG |
| | | CCCGTGTTGGGTGCCCTGTTCATCGGTGTGGCTGTGGCC |
| | | CCAGCTAACGACATCTACAACGAGCGCGAGCTGCTGAA |
| | | CAGCATGGGCATCAGCCAGCCCACCGTCGTATTCGTGAG |
| | | CAAGAAAGGGCTGCAAAAGATCCTCAACGTGCAAAAGA |
| | | AGCTACCGATCATACAAAAGATCATCATCATGGATAGCA |
| | | AGACCGACTACCAGGGCTTCCAAAGCATGTACACCTTCG |
| | | TGACTTCCCATTTGCCACCCGGCTTCAACGAGTACGACT |
| | | TCGTGCCCGAGAGCTTCGACCGGGACAAAACCATCGCCC |
| | | TGATCATGAACAGTAGTGGCAGTACCGGATTGCCCAAGG |
| | | GCGTAGCCCTACCGCACCGCACCGCTTGTGTCCGATTCA |
| | | GTCATGCCCGCGACCCCATCTTCGGCAACCAGATCATCC |
| | | CCGACACCGCTATCCTCAGCGTGGTGCCATTTCACCACG |
| | | GCTTCGGCATGTTCACCACGCTGGGCTACTTGATCTGCG |
| | | GCTTTCGGGTCGTGCTCATGTACCGCTTCGAGGAGGAGC |
| | | TATTCTTGCGCAGCTTGCAAGACTATAAGATTCAATCTG |
| | | CCCTGCTGGTGCCCACACTATTTAGCTTCTTCGCTAAGAG |
| | | CACTCTCATCGACAAGTACGACCTAAGCAACTTGCACGA |
| | | GATCGCCAGCGGCGGGGCGCCGCTCAGCAAGGAGGTAG |
| | | GTGAGGCCGTGGCCAAACGCTTCCACCTACCAGGCATCC |
| | | GCCAGGGCTACGGCCTGACAGAAACAACCAGCGCCATT |
| | | CTGATCACCCCCGAAGGGGACGACAAGCCTGGCGCAGT |
| | | AGGCAAGGTGGTGCCCTTCTTCGAGGCTAAGGTGGTGGA |
| | | CTTGGACACCGGTAAGACACTGGGTGTGAACCAGCGCG |
| | | GCGAGCTGTGCGTCCGTGGCCCCATGATCATGAGCGGCT |
| | | ACGTTAACAACCCCGAGGCTACAAACGCTCTCATCGACA |
| | | AGGACGGCTGGCTGCACAGCGGCGACATCGCCTACTGG |
| | | GACGAGGACGAGCACTTCTTCATCGTGGACCGGCTGAAA |
| | | AGCCTGATCAAATACAAGGGCTACCAGGTAGCCCCAGC |
| | | CGAACTGGAGAGCATCCTGCTGCAACACCCCAACATCTT |
| | | CGACGCCGGGGTCGCCGGCCTGCCCGACGACGATGCCG |
| | | GCGAGCTGCCCGCCGCAGTCGTCGTGCTGGAACACGGTA |
| | | AAACCATGACCGAGAAGGAGATCGTGGACTATGTGGCC |
| | | AGCCAGGTTACAACCGCCAAGAAGCTGCGCGGTGGTGTT |
| | | GTGTTCGTGGACGAGGTGCCTAAAGGACTGACCGGCAA |
| | | GTTGGACGCCCGCAAGATCCGCGAGATTCTCATTAAGGC |
| | | CAAGAAGGGCGGCAAGATCGCCGTGTAA |

### Embodiment 2: Effect of Introducing miR-122 Binding Sites at Different Positions in HBB 3′ UTR on the Stability of Fluc mRNA

### 1.1 UTR₁₂₂ Sequence Design and Gene Synthesis Based on HBB 3′UTR

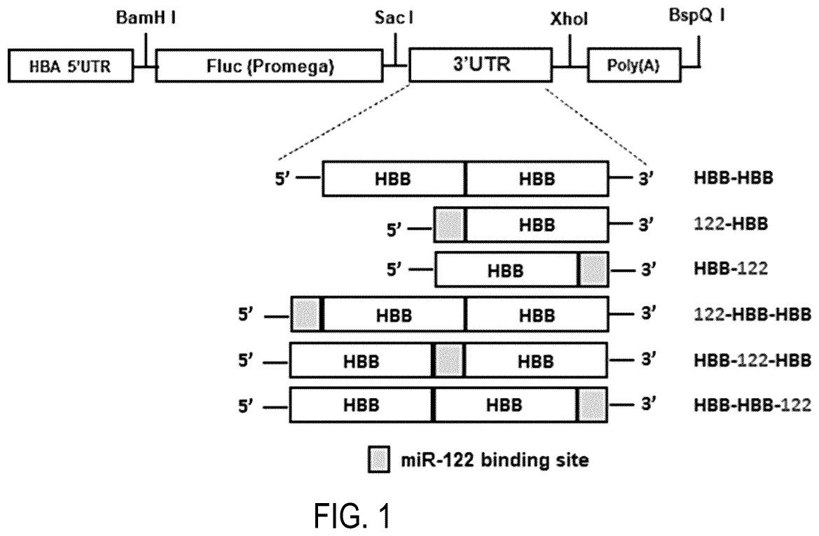

To test the positional effect of the binding site where miR-122 introduced into 3′ UTR, the sequence composition was first referred to: the 3′ UTR sequence (SEQ ID NO:2) of human hemoglobin β subunit (HBB) was selected, which belongs to one of the most efficient mammalian mRNA sequences for translation; different 3′ UTRs were connected to the firefly luciferase (Fluc) reporter gene vector. The expression level of genes was indirectly reflected by detecting the fluorescence intensity generated by the reaction between luciferase and substrate, in order to determine the effect of different 3′ UTR lengths on translation efficiency. Based on HBB 3′ UTR, 6 comparative plans were designed. The sequence design as set forth in FIG. 1, and the specific sequences can be found in SEQ ID NO:3-SEQ ID NO:8. The plasmid was synthesized by GenScript Biotech Corporation. The template plasmid for In Vitro Transcription (IVT) contained T7 promoter, HBA1-5′ UTR, Fluc-CDS, 3′ UTR, and segmented Poly (A) elements. Different 3′ UTRs were inserted at Sac I and Xho I enzyme cleavage sites, BspQ I was used as a linearized enzyme cleavage site.

### 1.2 Selecting Appropriate Cell Lines for In Vitro Evaluation

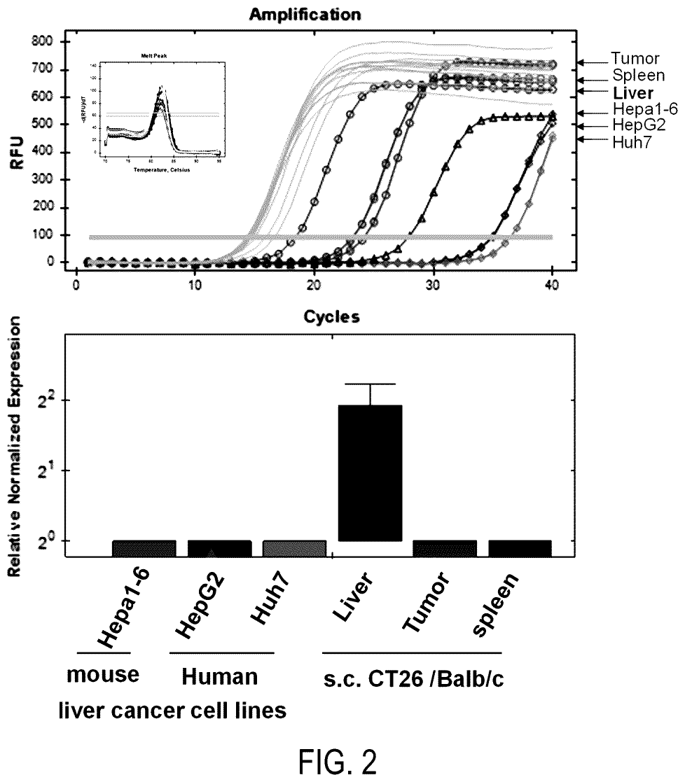

Literature has reported that miR-122 was specifically highly expressed in normal liver tissue but at a low level in tumor cells. q-PCR was used to detect the expression of endogenous miR-122 in human liver cancer cell lines HepG2 and Huh-7, and mouse liver cancer cell line HepG1-6; and the liver tissue of 7-8 week old female Balb/c colon cancer CT26 subcutaneous tumor bearing mice was used as a positive control, while the spleen and tumor of the mice were used as negative controls. As shown in FIG. 2, miR-122 was only highly expressed in mouse liver tissue, with low expression in spleen and tumors, while miR-122 was expressed as low as near baseline in human liver cancer cell lines HepG2 and Huh-7 and mouse liver cancer cell line HepG1-6. Therefore, HepG2, Huh-7, and HepG1-6 cell lines can all be used to evaluate the response of different 3′ UTR₁₂₂ to miR-122 in vitro. In subsequent in vitro evaluations, we selected Huh-7 cells with the highest transfection efficiency.

1.3 Cell Experiments to Observe the Responsiveness of Different HBB UTR₁₂₂ to miR-122 Mimics

miR-122 has two mature forms, one of which is hsa-miR-122-5p with an Accession number of MIMAT0000421; the other is hsa-miR-122-3p, with an Accession number of MIMAT000 4590. hsa-miR-122-5p (CCUUACGAGUGUGGAGUGACAAUGGUGUCUAAACUCAAACGCCAUUAUCACUAA AUAGCUACUGCUAGGC, SEQ ID NO:21) was synthesized as miR-122 mimics. The different plasmids shown in FIG. 2 were enzymatically digested (BspQ I) to obtain linearized templates, and then purified by IVT to obtain Fluc mRNA with different 3′ UTRs. 1 μg mRNA was co-transfected with different doses of miR-122 mimics (0-25 nM) into Huh7 cells. After 24 hours, the cells were lysed and Luciferase substrate was added. Fluc activity was detected by a multi-label microplate detector to indicate the translation efficiency of Fluc mRNA, and to analyze the reactivity of different UTR₁₂₂ to miR-122.

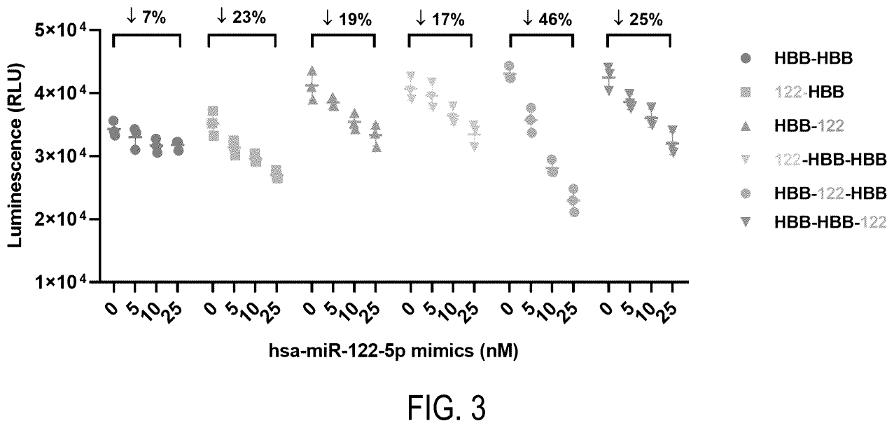

As shown in FIG. 3, miR-122 mimics inhibited the expression of all Fluc mRNA containing miR-122 binding sites in a dose-dependent manner, and showed significant differences in HBB UTR122 positional effects. From the maximum dose-response inhibition rate data, the structural design of HBB-122-HBB was optimal (46% relative to baseline inhibition), followed by HBB-HBB-122 (25%). Both of them had high baseline (Fluc activity when not transfected with mimics), suggesting that the design scheme of placing the miR-122 binding site sequence between two 3′ UTR elements had the best inhibitory effect on miR-122. This may provide an optimal 3′ UTR design strategy for reducing the expression of mRNA drugs in the liver using miR-122, and it is also a reference for the integration design of other miRNA binding sites in the 3′ UTR.

2 Confirm that it has the Best Effect when the miR-122 Binding Site is Located in the Middle of the Double Copy UTR Element
2.1 UTR122 Sequence Design and Gene Synthesis Based on AES and mtRNR1 3′ UTR

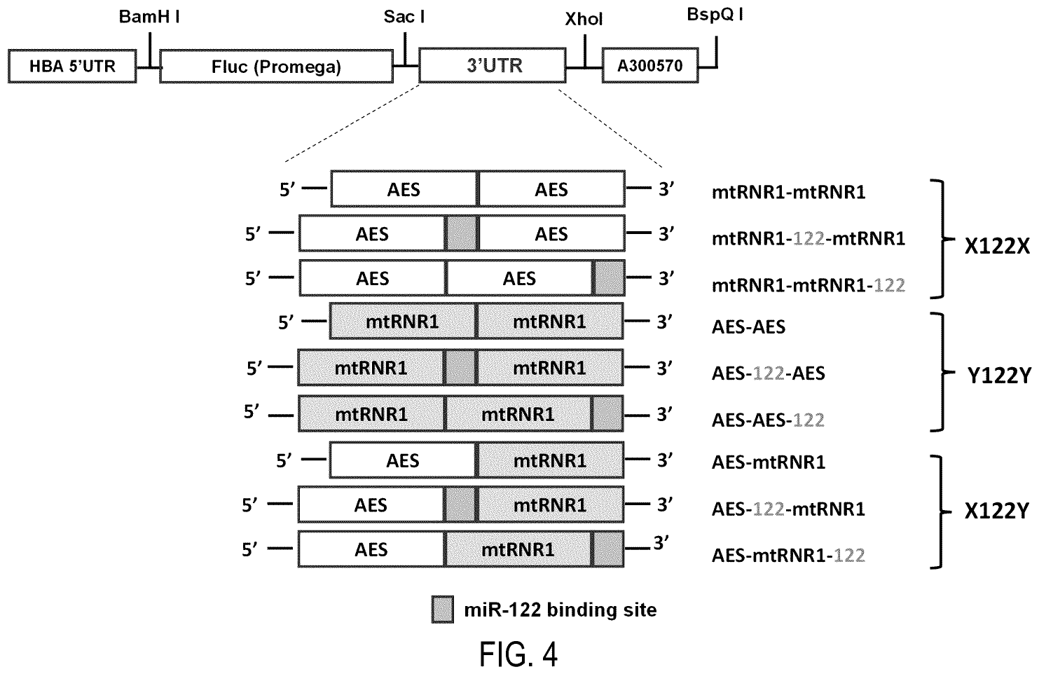

It was previously found that the insertion of the miR-122 binding site into the middle of the double copy HBB element has the best mRNA inhibitory regulation effect. To further prove that the design strategy of introducing miR-122 binding site into the middle of the double copy UTR element is optimal, the AES and mtRNR1 3′ UTR elements used by BioNTech in the COVID-19 mRNA vaccine BNT162b were selected. The X122X, Y122Y and X122Y with the miR-122 binding site located between two identical or different 3′ UTRs were designed as experimental embodiments, and the XX122, YY122 and XY122 with the miR-122 binding site located at the ends of two identical or different 3′ UTRs were selected as comparative examples, and they were compared and verified by cell experiments. As described in 1.1, a total of 9 sequences were designed as shown in FIG. 4. The specific sequences are shown in SEQ ID NO:10-SEQ ID NO:19. A Fluc reporter plasmid for IVT was constructed, and synthesized by GenScript Biotech Corporation.

2.2 Observation of the Reactivity of Different AES and mtRNR UTR₁₂₂ Towards miR-122 Mimics Through Cell Experiments

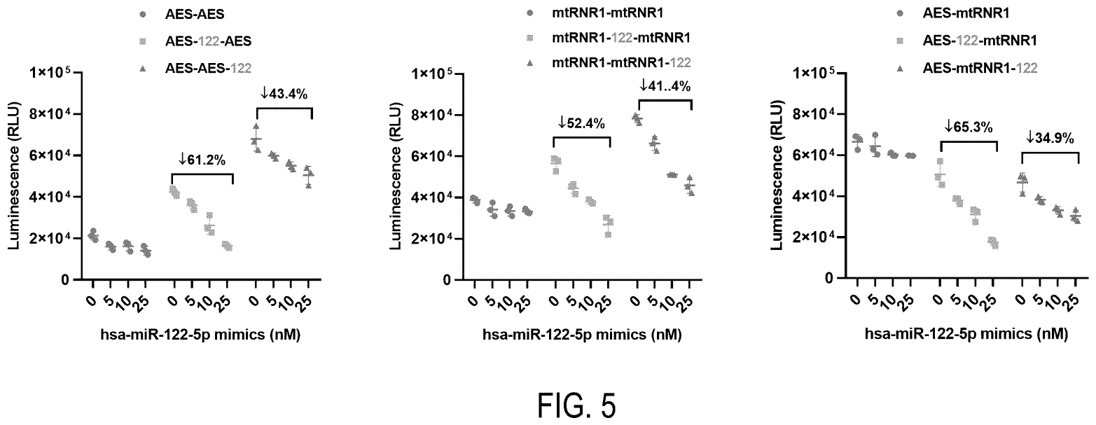

According to 1.3, different mRNAs as shown in FIG. 4 were co-transfected with miR-122 mimics for 24 hours and Fluc activity was analyzed afterwards; the results were shown in FIG. 5, although the Fluc baseline varied after introducing miR-122 binding sites into different UTRs, the inhibition rates of miR-122 mimics by different UTR₁₂₂ showed that AES-122-AES>AES-AES-122, mtRNR1-122-mtRNR1>mtRNR1-mtRNR1-122, AES-122-mtRNR1>AES-mtRNR1-122. The effect of miR 122 mimics was significantly better when the miR-122 binding site was placed in the middle of the double copy 3′ UTR element than when at the end position. This was consistent with the screening results based on HBB UTR₁₂₂, proving that the design strategy X122X was superior to XX122, and X122Y was superior to XY122, which was also applicable to the application of 3′ UTR to other miRNA binding sites in mRNA drug design.

2.3 Different Responses of UTR₁₂₂ to miR-122 Mimics in Different Cells

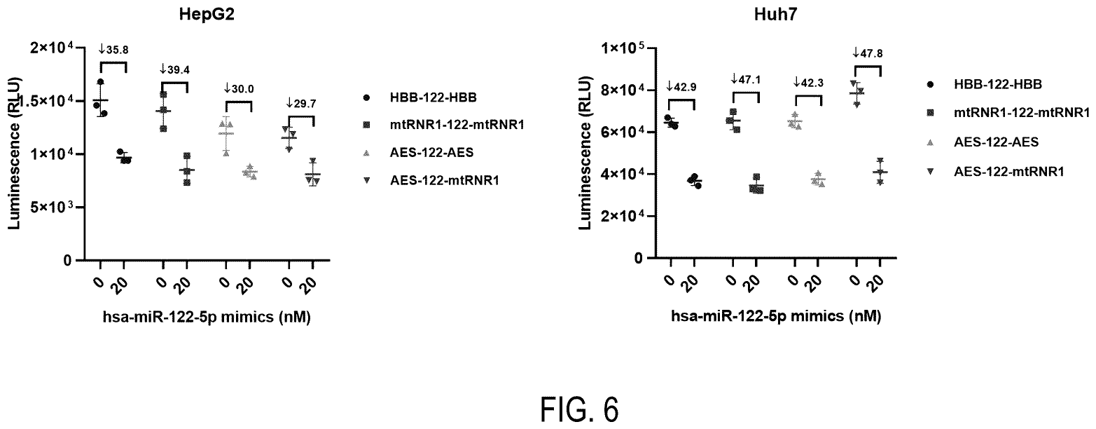

The regulation of mRNA stability and translation efficiency by 3′ UTR varied in different physiological and cellular states. It was observed that the four optimal UTR₁₂₂ (HBB-122-HBB, AES-122-AES, mtRNR1-122-mtRNR1, and AES-122-mtRNR1) screened earlier had slightly different translation regulation of Fluc mRNA and response inhibition to miR-122 mimics in human liver cancer cell lines HepG2 and Huh7. As shown in FIG. 6, in HepG2 cells, Fluc mRNA carrying HBB-122-HBB and mtRNR1-122-mtRNR1 showed the highest luciferase activity and the best response to miR-122 mimics, while AES-122-mtRNR1 showed the worst performance; but in Huh7 cells, four UTR₁₂₂ showed similar performance, and AES-122-mtRNR1 was slightly superior. This suggested that although the effects of 3′ UTR sequences on mRNA stability and translation varied in different cells, the analysis of microRNA responding to specific 3′ UTR carrying microRNA binding sites is not significantly different in different cells.

In summary, 3′ UTR₁₂₂ of different designs were inserted into luciferase reporter gene vectors using luciferase reporter gene vectors. In vitro co-transfection experiments of mRNA and miR-122 mimics were conducted to detect the fluorescence intensity generated by the reaction between luciferase and substrate, indirectly reflecting the expression level of Fluc and analyzing the expression inhibition effect, so as to determine the optimal 3′ UTR₁₂₂ design strategy. The screening results showed that introducing the miR-122 binding site between the double copy 3′ UTR element could achieve the most effective specific response inhibition of miR-122. On the other hand, the selection of UTR elements may need to be determined based on specific applications, but the genes of human α- and β-globin are naturally highly expressed ones and are still the ideal choices for mRNA 3′ UTR in most cases, which can be further enhanced by the use of double copy β-globin 3′ UTR (2XHBB).

### Embodiment 3: In Vivo Experiment for Verification of Fluc-2XHBB₁₂₂ Non-Hepatic Translation Efficiency in Mice

Currently, the tissue targeting of LNP is mainly limited to the liver. We used microfluidic technology to produce LNP particles with a diameter of approximately 100 nm, and encapsulated Fluc mRNA carrying HBB-122-HBB or HBB-HBB 3′ UTR into LNP nanoparticles to obtain test samples named Fluc-2XHBB₁₂₂/LNP and Fluc-2XHBB/LNP, respectively. Through intravenous injection (i.v.), subcutaneous injection (s.c.), and intratumoral injection (i.Tu), it was verified that Fluc-2XHBB₁₂₂/LNP carrying HBB-122-HBB had the characteristic of reducing FLuc mRNA expression in the liver compared with that carrying Fluc-2XHBB/LNP. This proved that inserting miR-122 binding site between the double copy 3′ UTR could achieve effective non-hepatic delivery expression of mRNA in vivo.

### 3.1 Intravenous Injection

Female BALB/c mice aged 7-8 weeks were divided into two groups based on body weight: Fluc-2XHBB/LNP group, Fluc-2XHBB₁₂₂. (n=3). The in vivo bioluminescence signals in the abdominal and lateral directions of each mouse using an intravenous injection live imaging system was observed.

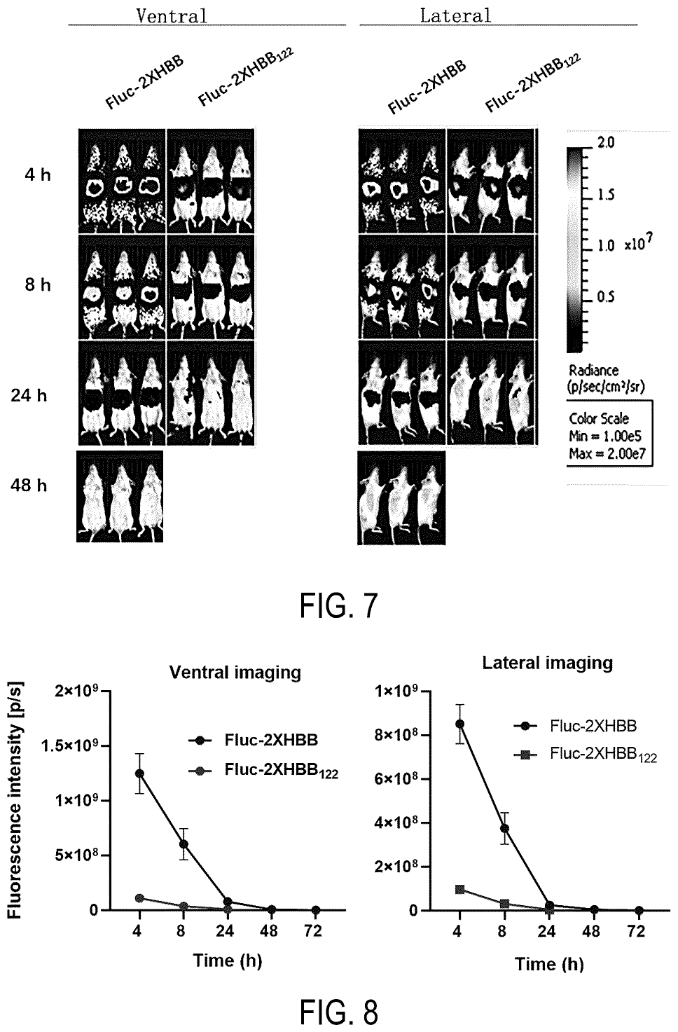

The results were shown in FIG. 7, the bioluminescence signals of mice in each group were mainly distributed in the liver area 4 hours after drug injection, and gradually weakened over time. The fluorescence signal of the Fluc-2XHBB/LNP group disappeared after 48 hours, and the fluorescence signal of the Fluc-2XHBB₁₂₂/LNP group lasted for less than 24 hours; wherein the bioluminescence signal values of the Fluc-2XHBB₁₂₂/LNP group were significantly lower than those of the Fluc-2XHBB/LNP group at each time point (p<0.001) (as shown in FIG. 8). It can be seen that under intravenous administration, compared with the Fluc-2XHBB/LNP group, the stability of Fluc mRNA delivered by Fluc-2XHBB₁₂₂/LNP group was lower. Carrying 2XHBB₁₂₂ effectively reduced mRNA expression in the liver, and mRNA drugs were more easily degraded in hepatocytes.

### 3.2 Subcutaneous Injection

Female BALB/c mice aged 7-8 weeks were divided into two groups based on body weight: Fluc-2XHBB₁₂₂/LNP (i.v.) group and Fluc-2XHBB₁₂₂/LNP (s.c.) group (n=3), with a dosage of 10 μg per mouse in each group. At 4 hours, 8 hours, and 24 hours after administration, in vivo imaging was performed, and small animal imaging system was used to observe the in vivo bioluminescence signals of mice in the ventral and lateral directions after subcutaneous administration.

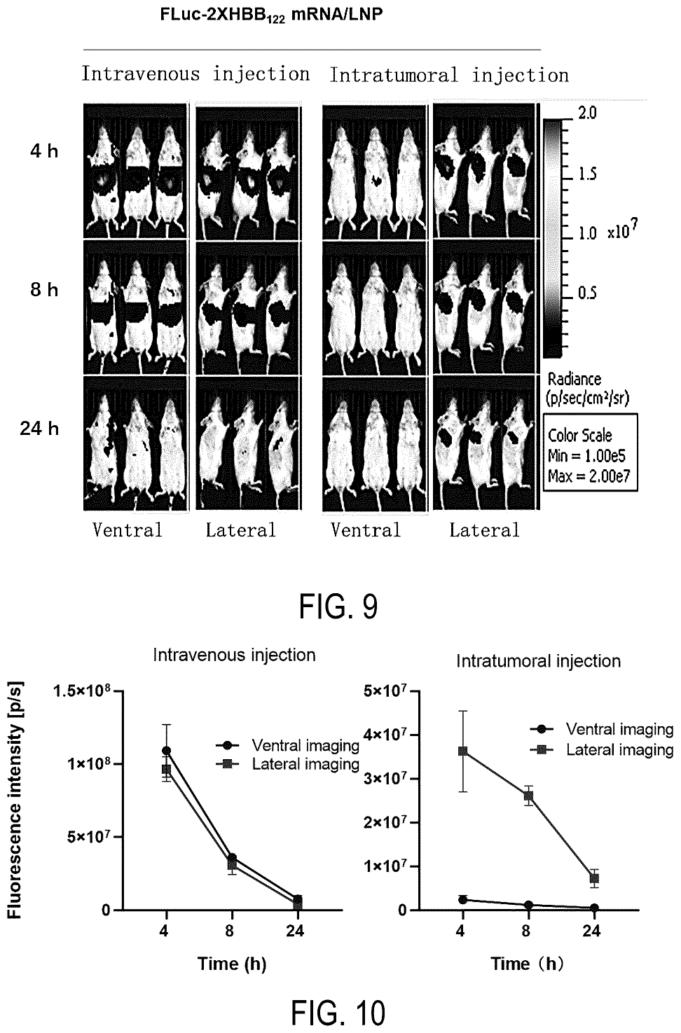

The results were shown in FIG. 9, the bioluminescence signals of mice in each group 4 hours after administration were mainly distributed in the liver area, and the signals gradually attenuated over time; the ventral fluorescence signals of the Fluc-2XHBB₁₂₂/LNP (i.v.) group were close to those of the lateral imaging, while the ventral fluorescence signals of the Fluc-2XHBB₁₂₂/LNP (s.c.) group were significantly lower than those of the lateral imaging (FIG. 10). Because abdominal imaging can fully expose the fluorescence signal of the liver, subcutaneous injection of Fluc-2XHBB₁₂₂/LNP significantly reduced the liver signal, but the signal at the injection site was higher, and the fluorescence signal was maintained for more than 24 hours. Therefore, Fluc-2XHBB₁₂₂/LNP could effectively reduce mRNA drugs expression in the liver under subcutaneous injection.

### 3.3 Intratumoral Injection

A subcutaneous MC38 tumor bearing model was constructed in 7-8 week old female C57BL/6 mice. When the tumor volume reached 180 mm3, the mice were divided into two groups according to the tumor volume for intratumoral injection administration, namely: Fluc-2XHBB/LNP group and Fluc-2XHBB₁₂₂/LNP group (n=3). The dosage was 5 μg per mouse for each group, and in vivo imaging was performed 6 hours after administration. The small animal in vivo imaging system was used to observe the in vivo bioluminescence signals of mice in the ventral and subcutaneous administration directions, and perform in vitro imaging of mouse tumors and livers.

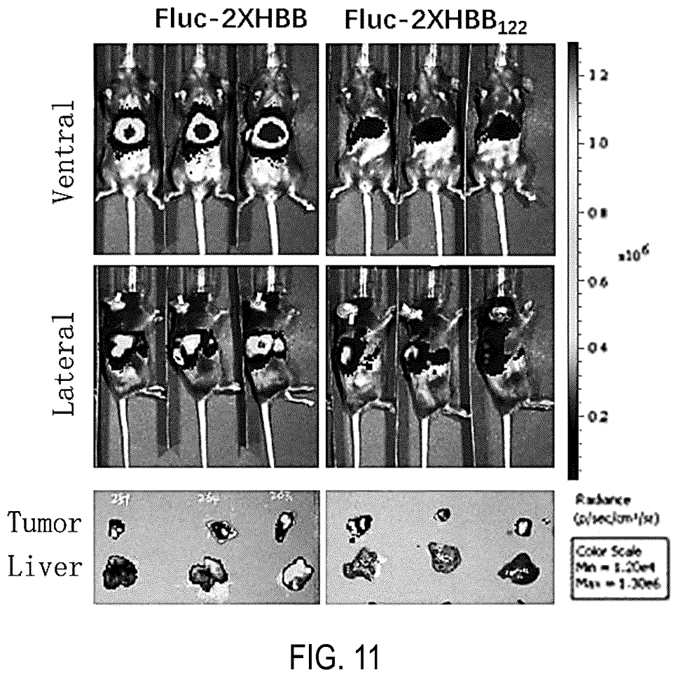

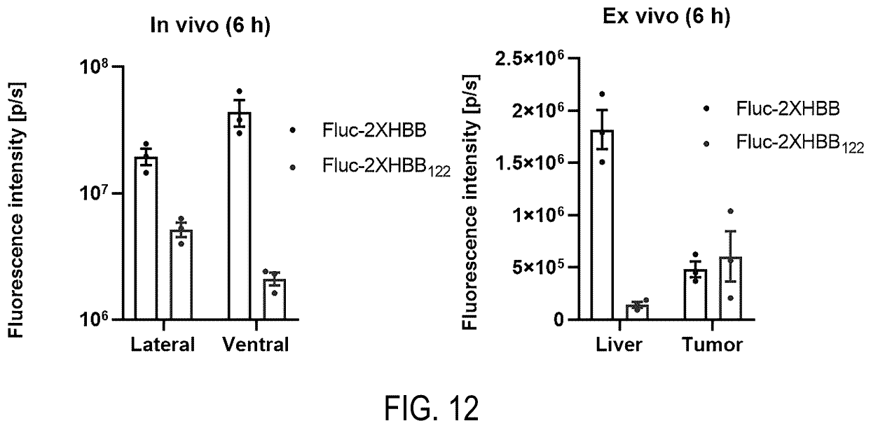

As shown in FIG. 11, after 6 hours of administration, the bioluminescence signals of mice in each group were mainly distributed in the tumor and liver regions. The abdominal and lateral imaging fluorescence signals of the Fluc-2XHBB₁₂₂/LNP group were lower than those of the Fluc-2XHBB/LNP group. The luminescence signal of the tumor site was stronger in the Fluc-2XHBB₁₂₂/LNP group, while the luminescence signal of the liver site was weaker; the tumor and liver regions in the Fluc-2XHBB/LNP group showed strong signals, and the luminescence signal intensity of the tumor sites of the two groups was the same. However, the luminescence signal of the liver region in the Fluc-2XHBB/LNP group was 13-fold higher than that of the Fluc-2XHBB₁₂₂/LNP group (FIG. 12).

It can be seen that under intratumoral administration, compared with the Fluc-2XHBB/LNP group, the Fluc-2XHBB₁₂₂/LNP group greatly reduced the expression of delivered mRNA drugs in the liver site, while not affecting the expression of mRNA drugs in the tumor site.

### 3.4. Validation by Synthesizing GFP-2XHBB₁₂₂ Sequence in Normal Cell Lines

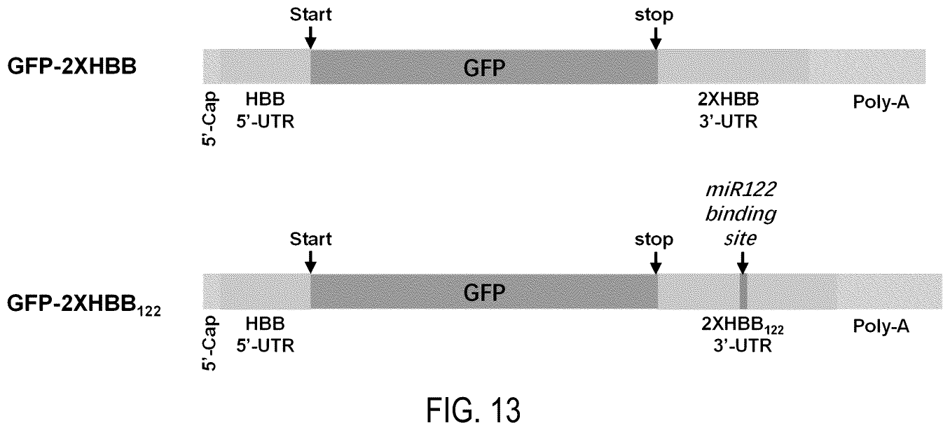

Previously, we used a luciferase reporter gene vector to screen for the optimal UTR₁₂₂ in human liver cancer cell lines, and confirmed through in vivo imaging in mice that 2XHBB₁₂₂ reduced mRNA drugs expression in the liver without affecting its injection site (subcutaneous injection site and tumor) expression. In addition, we used the green fluorescent protein gene GFP to connect 2XHBB₁₂₂ to the GFP gene vector (FIG. 13), transfected the vector into normal 293T cell line and CHO cell line, and observed by fluorescence microscopy or flow cytometry analysis, which can also effectively determine the negative regulatory effect of miR-122 binding site mediated 3′ UTR on GFP mRNA.

### 3.4.1 Transfection of 293T Cells

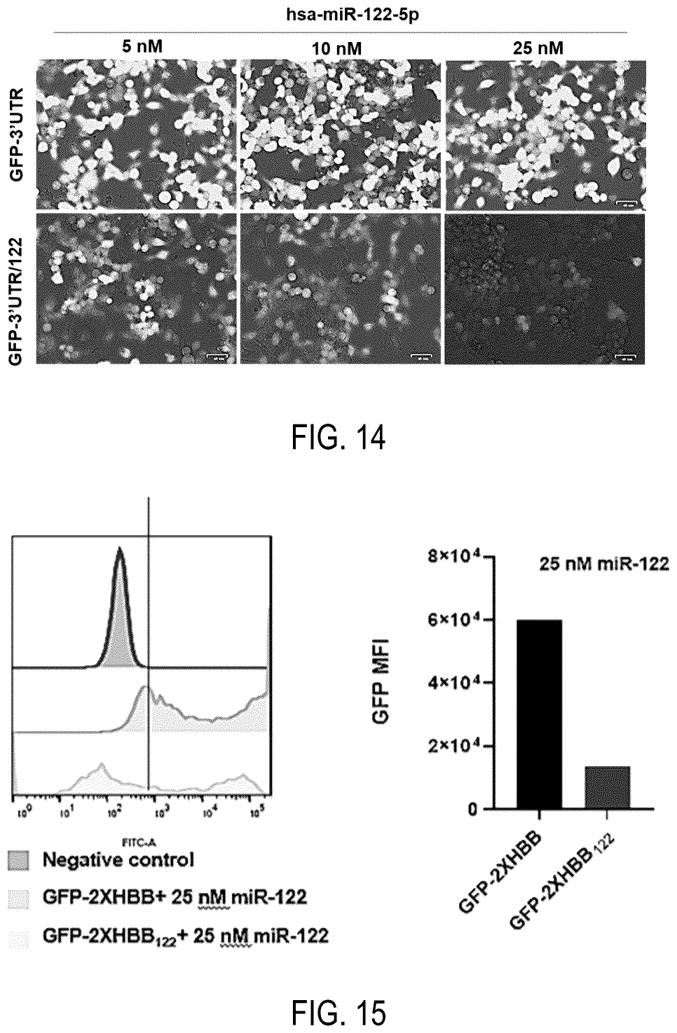

Following the aforementioned method, the image of GFP-2XHBB and GFP-2XHBB₁₂₂ mRNA as shown in FIG. 13 was obtained through IVT. 1 μg of mRNA was co-transfected with nM, 10 nM, and 25 nM miR-122 mimics using nucleic acid transfection reagents into 293T cells. Fluorescence microscopy observation showed that as miR-122 mimics increased, there was no significant change in GFP fluorescence intensity in 293T cells transfected with GFP-2XHBB mRNA, while GFP intensity gradually decreased in 293T cells transfected with GFP-2XHBB₁₂₂ mRNA with the increase of miR-122 mimics content (FIG. 14). The GFP expression level (MFI, average fluorescence intensity) in 293T cells was detected by flow cytometry with the addition of nM miR-122 mimics. It was significantly lower in GFP-2XHBB₁₂₂ transfected cells than that in GFP-2XHBB transfected cells (FIG. 15).

### 3.4.2 Transfection of CHO Cells

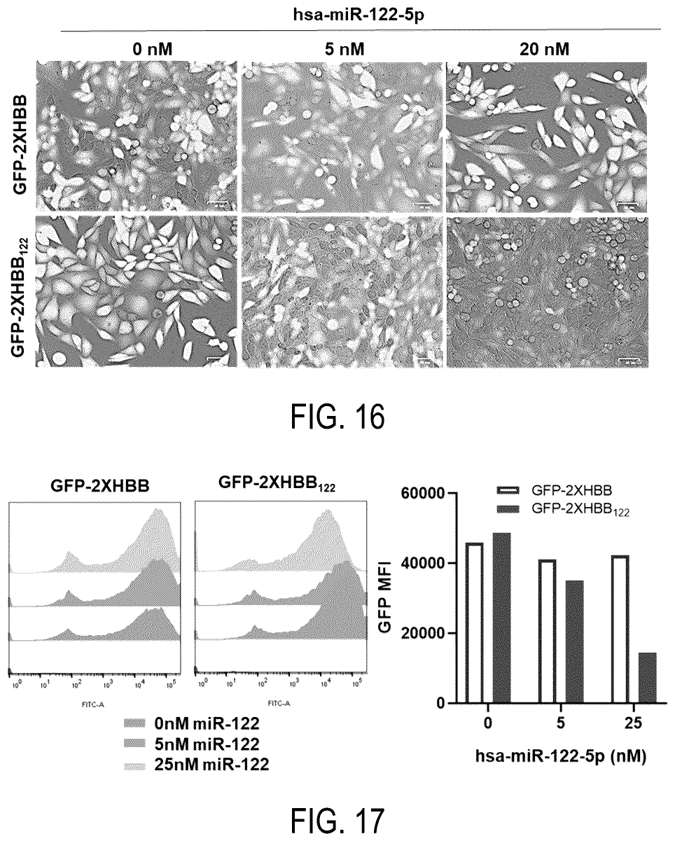

The co-transfection results of GFP-2XHBB or GFP-2XHBB₁₂₂ mRNA with different doses of miR-122 mimicsin into CHO cells were consistent with transfection into 293T cells. As shown in FIG. 16, observed through fluorescence microscopy, with the increase of miR-122 mimics content, the GFP fluorescence intensity in CHO cells transfected with GFP-2XHBB₁₂₂ mRNA gradually decreased, while the GFP fluorescence intensity in CHO cells transfected with GFP-2XHBB mRNA showed no significant changes. The flow cytometry results also showed that the GFP signal in CHO cells transfected with GFP-2XHBB₁₂₂ mRNA gradually decreased with the increase of miR-122 mimics content, while there were no significant changes in CHO cells transfected with GFP-2XHBBmRNA.

The above in vitro cell experiments based on GFP gene demonstrated that the introduction of miR-122 binding site at the middle position of double copy HBB 3′ UTR effectively reduced mRNA drug expression in cells containing a large amount of miR-122.

The embodiments above merely express several implementations of the present disclosure. The descriptions of the embodiments are relatively specific and detailed, but may not therefore be construed as the limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. An mRNA drug, which is less expressed in the liver after in vivo delivery, comprising mRNA and a drug carrier, wherein a 3′ UTR component of the mRNA comprises two identical or different 3′ UTR sequences, and a miR-122 binding site is inserted between the two 3′ UTR sequences.

2. The mRNA drug according to claim 1, wherein the 3′ UTR sequence is at least one of the 3′ UTR sequence of human hemoglobin β subunit, AES element, and mtRNR1 element.

3. The mRNA drug according to claim 2, wherein the 3′ UTR component is composed of the AES element, the miR-122 binding site, and the AES element.

4. The mRNA drug according to claim 2, wherein the 3′ UTR component is composed of the AES element, the miR-122 binding site, and the mtRNR1 element.

5. The mRNA drug according to claim 2, wherein the 3′ UTR component is composed of the mtRNR1 element, the miR-122 binding site, and the mtRNR1 element.

6. The mRNA drug according to claim 2, wherein the 3′ UTR component is composed of the 3′ UTR sequence of the human hemoglobin β subunit, the miR-122 binding site, and the 3′ UTR sequence of the human hemoglobin β subunit.

7. The mRNA drug according to claim 1, wherein the sequence of the miR-122 binding site as set forth in SEQ ID NO: 1.

8. The mRNA drug according to claim 2, wherein the 3′ UTR sequence of the human hemoglobin β subunit as set forth in SEQ ID NO: 2.

9. The mRNA drug according to claim 2, wherein the AES element as set forth in SEQ ID NO: 9.

10. The mRNA drug according to claim 2, wherein the mtRNR1 element as set forth in SEQ ID NO: 13.

11. The mRNA drug according to claim 1, wherein the drug carrier is a lipid nanoparticle, a complex and a polymer nanoparticle, an exosome, or a biological microvesicle.

12. The mRNA drug according to claim 1, is an mRNA vaccine.

13. A method for preparing an mRNA drug, wherein the mRNA drug comprises mRNA and a drug carrier, comprising following steps: including two identical or different 3′ UTR sequences in a 3′ UTR component of mRNA, inserting a miR-122 binding site between connections of the two 3′ UTR sequences, and obtaining an mRNA for preparing an mRNA drug.

14. A method for reducing an expression of an mRNA drug in a liver after in vivo delivery, wherein a 3′ UTR component of mRNA in the mRNA drug comprises two identical or different 3′ UTR sequences, and a miR-122 binding site is inserted between the two 3′ UTR sequences.

15. The method according to claim 14, wherein the sequence of the miR-122 binding site as set forth in SEQ ID NO: 1.

16. The method according to claim 14, wherein the 3′ UTR sequence is at least one of the 3′ UTR sequence of human hemoglobin β subunit, AES element, and mtRNR1 element.

17. The method according to claim 14, wherein the 3′ UTR component is composed of the AES element, the miR-122 binding site, and the AES element; or
the 3′ UTR component is composed of the AES element, the miR-122 binding site, and the mtRNR1 element; or
the 3′ UTR component is composed of the mtRNR1 element, the miR-122 binding site, and the mtRNR1 element; or
the 3′ UTR component is composed of the 3′ UTR sequence of the human hemoglobin subunit, the miR-122 binding site, and the 3′ UTR sequence of the human hemoglobin β subunit.

18. The method according to claim 16, wherein the 3′ UTR sequence of the human hemoglobin β subunit as set forth in SEQ ID NO: 2.

19. The method according to claim 16, wherein the AES element as set forth in SEQ ID NO: 9.

20. The method according to claim 16, wherein the mtRNR1 element as set forth as SEQ ID NO: 13.
